(19) United States
(12) Patent Application Publication
(10) Pub. No.: US 2025/0228593 A1
(43) Pub. Date: Jul. 17, 2025

(54) Fixation System for Restoring a Posterior Tension Band in a Spine of a Patient

(71) Applicant: Graham Mouw, Miami Beach, FL (US)

(72) Inventor: Graham Mouw, Miami Beach, FL (US)

(21) Appl. No.: 18/414,267

(22) Filed: Jan. 16, 2024

(51) Int. Cl. A61B 17/70; A61B 17/02; A61B 17/88

(52) U.S. Cl. CPC A61B 17/7022; A61B 17/025; A61B 17/7053; A61B 17/7067; A61B 17/8869; A61B 2017/0256

(57) ABSTRACT

The present devices and methods provide a fixation system for restoring a posterior tension band in a patient. A first bracket is mounted to a first posterior boney landmark of a first lamina-spinous process; and a second bracket is mounted to a second posterior boney landmark of a second lamina-spinous process. A flexible tensioning cord is fastened between the first bracket and the second bracket, tensioned to induce lordosis in the lumbar spine; less tension would maintain kyphosis in the thoracic spine. A system for implanting the assembly includes a tensioning tool having a bracket grasping jaw half pivotally opposing a cord grasping jaw half, where their separation tensions the flexible tensioning cord while the cord is being coupled to one or more brackets. Advantageously, this permits restoration of the lumbar or thoracic posterior tension band in a motion sparing (non-fusion) manner and allows complete removal of the lamina/spinous process at a single or multiple levels.

### BACKGROUND

The subject of this patent application relates generally to medical methods and apparatus, and more particularly, to devices for restoring a posterior tension band in a patient.

By way of background, lumbar laminectomies (removal of lamina) are one of the most common of all spine procedures. However, removal of complete lamina (laminectomy) or partial lamina (hemilaminotomy or hemilaminectomy) creates inherent instability between consecutive or non-consecutive lumbar segments. This is particularly true in the setting of spondylolisthesis, where one vertebral body of a spinal level is not in alignment with the adjacent vertebral body. In spondylolisthesis, there is already some spinal instability and further removal of lamina would likely destabilize an existing spondylolisthesis. Currently lumbar fixation (fusion) is the predominant option with pedicle screws or interspinous rigid devices to maintain stability.

Aspects of the present invention fulfill these needs and provide further related advantages as described in the following summary.

### SUMMARY

Aspects of the present invention teach certain benefits in construction and use which give rise to the exemplary advantages described below.

The present specification discloses a fixation system for restoring a posterior tension band in a spine of a patient, the fixation system having a flexible tensioning cord, a first lamina-spinous process bracket, a second lamina-spinous process bracket, and a tensioning tool. The flexible tensioning cord has a first cord portion, a second cord portion, and a connecting portion extending between the first cord portion and the second cord portion; the first lamina-spinous process bracket has a first anchoring portion and a first cord securing portion adapted to permit the flexible tensioning cord to slide therethrough in an open configuration and adapted to securely grasp the flexible tensioning cord at the first cord portion in a locked configuration, the first lamina-spinous process bracket adapted to be anchored to a first posterior boney landmark of the spine by the first anchoring portion; the second lamina-spinous process bracket has a second anchoring portion and a second cord securing portion adapted to permit the flexible tensioning cord to slide therethrough in the open configuration and adapted to securely grasp the flexible tensioning cord at the second cord portion in the locked configuration, the second lamina-spinous process bracket adapted to be anchored to a second posterior boney landmark of the spine by the second anchoring portion; and the tensioning tool having a bracket grasping jaw half opposing a cord grasping jaw half. When in a tensioning configuration, the first lamina-spinous process bracket is in the locked configuration, the second lamina-spinous process bracket is in the open configuration, the bracket grasping jaw half of the tensioning tool is releasably coupled to the first lamina-spinous process bracket and the cord grasping jaw half is releasably coupled to the flexible tensioning cord to hold the connecting portion in tension while the second lamina-spinous process bracket is reconfigured to the locked configuration, holding therebetween the flexible tensioning cord at a therapeutic tension.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is an assembled perspective view of an exemplary embodiment of the present fixation system;

FIG. 2 is an exploded perspective view of the fixation system of FIG. 1;

FIG. 3 is a perspective view of a patient's spine, illustrating lateral holes being drilled through a plurality of posterior boney landmarks, in accordance with the present system and method;

FIG. 4 is an exploded rear view of the fixation system of FIG. 1, aligned with and ready for insertion through respective lateral holes of the posterior boney landmarks; and

FIG. 5 is a partially assembled rear view of the fixation system of FIG. 4, illustrating the lamina-spinous process brackets mounted to the respective posterior boney landmarks;

FIG. 6 is a partially assembled rear view of the fixation system of FIG. 5, illustrating the flexible tensioning cord in the process of being tensioned and coupled to the respective lamina-spinous process brackets;

FIG. 7 is a partially assembled rear view of the fixation system of FIG. 6, illustrating the flexible tensioning cord continuing in the process of being tensioned and coupled to the respective lamina-spinous process brackets;

FIG. 8 is a fully assembled rear view of the fixation system of FIG. 7, illustrating the flexible tensioning cord tensioned and coupled to the respective lamina-spinous process brackets;

FIG. 9 is a cross sectional of one of the fully assembled lamina-spinous process brackets of FIG. 8, taken at section 9-9; and

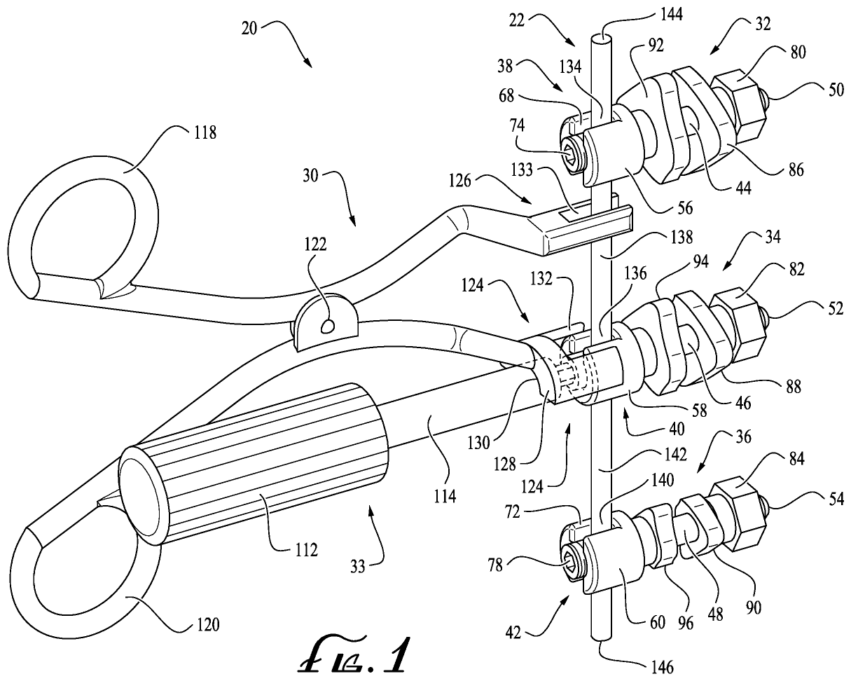

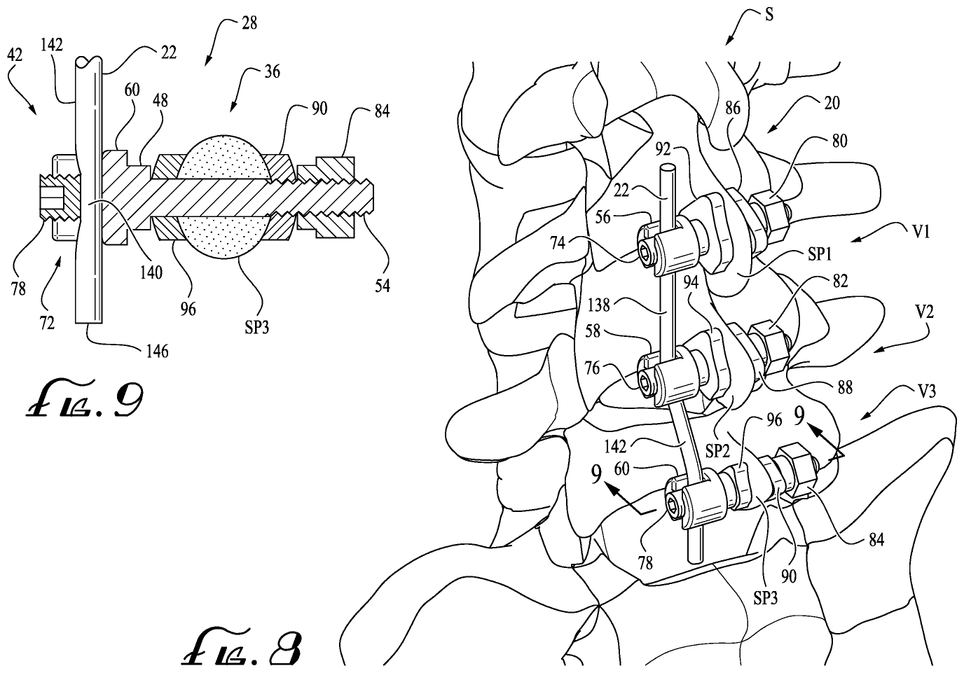

FIG. 8 is a fully assembled rear view of the fixation system of FIG. 1, illustrating two lamina-spinous process brackets mounted to two respective nonconsecutive laminas, with the flexible tensioning cord spanning a space on which a laminectomy has been performed.

The above-described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

The detailed descriptions set forth below in connection with the appended drawings are intended as a description of embodiments of the invention, and is not intended to represent the only forms in which the present invention may be constructed and/or utilized. The descriptions set forth the structure and the sequence of steps for constructing and operating the invention in connection with the illustrated embodiments. It is to be understood, however, that the same or equivalent structures and steps may be accomplished by different embodiments that are also intended to be encompassed within the spirit and scope of the invention.

The present devices and methods in one or more embodiments provide a fixation system for restoring a posterior tension band in a patient. A first lamina-spinous process bracket (also referred to herein as the first bracket) is mounted to a first posterior boney landmark of a first lamina; and a second lamina-spinous process bracket (also referred to herein as the second bracket) is mounted to a second posterior boney landmark of a second lamina. A flexible tensioning cord is fastened between the first lamina-spinous process bracket and the second lamina-spinous process bracket, tensioned to induce lordosis in the spine. A system for implanting the assembly includes a tensioning tool having a bracket grasping jaw half pivotally opposing a cord grasping jaw half, where their separation tensions the flexible tensioning cord while the cord is being coupled to one or more lamina-spinous process brackets. Advantageously, this permits restoration of the lumbar or thoracic posterior tension band in a motion sparing (non-fusion) manner and allows complete removal of the lamina/spinous process at a single or multiple levels. Further, the present system can also be used to correct some forms of scoliosis through a posterior column approach.

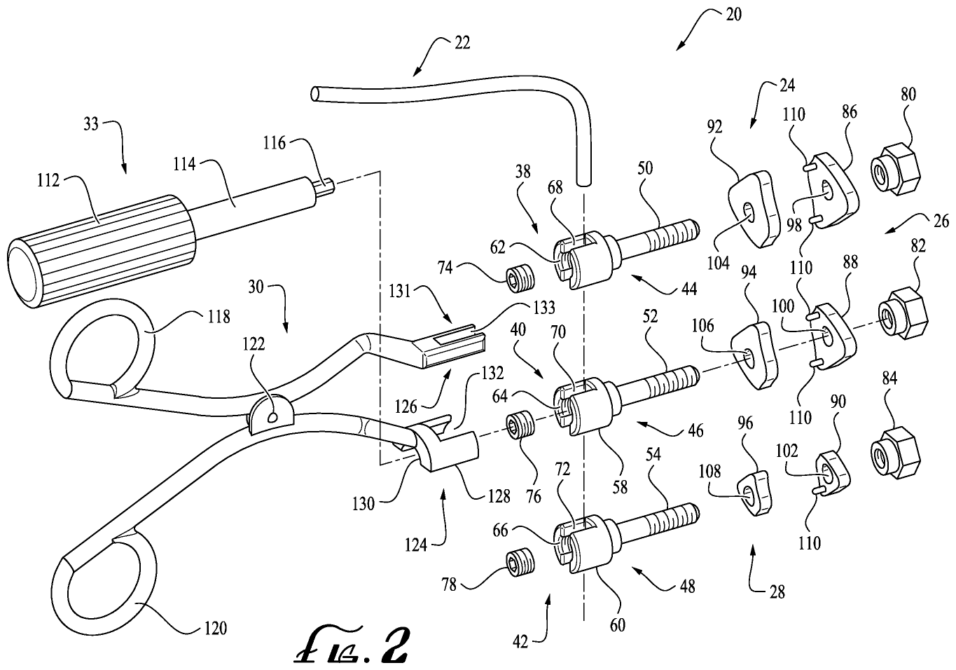

Looking first at FIGS. 1-2, an example embodiment of the present fixation system 20 is illustrated in assembled and exploded configurations, respectively. In one or more embodiments, the fixation system 20 includes a tensioning cord 22 extending in tension between a first lamina-spinous process bracket 24 and a second lamina-spinous process bracket 26, where the tensioning cord 22 is firmly coupled to the first lamina-spinous process bracket 24 by a first cord portion 134 at an anchoring portion 32 of the first lamina-spinous process bracket 24, and to the second lamina-spinous process bracket 26 by a second cord portion 136 at an anchoring portion 34 of the second lamina-spinous process bracket 26. Installation tools, such as a tensioning tool 30, a driver tool 33, and a drill D are utilized to implant the first lamina-spinous process bracket 24 and the second lamina-spinous process bracket 26 to a portion of a patient's spine S. Although, an example embodiment of the present fixation system 20 is described and illustrated as including installation/adjustment tools and multiple brackets 24, 26, etc., various parts of the system can include inventive aspects absent the remainder of the system. For example, an individual lamina-spinous process bracket 24, the tensioning tool 30, the method for and posterior location of the implant, and so on, can be claimed in isolation of other parts of the system 20.

Looking more closely at the broad construction of the first lamina-spinous process bracket 24, the second lamina-spinous process bracket 26, and the third lamina-spinous process bracket 28 (also referred to herein as the third bracket), each have an anchoring portion 32, 34, 36 (the portion of the lamina-spinous process brackets 24, 26, 28 that couples to a posterior boney landmark, such as the spinous process, lamina, or junction of the spinous process and lamina that are intact or have sufficient structure to accept a bracket 24, 26, 28). Further, the first lamina-spinous process bracket 24, the second lamina-spinous process bracket 26, and the third lamina-spinous process bracket 28 each have a cord securing portion 38, 40, 42 (the portion of the lamina-spinous process brackets 24, 26, 28 that couples to the flexible tensioning cord 22 at varying cord portions 134, 136, 140 along the length of the flexible tensioning cord 22).

Specifically referencing the illustrated assembly comprising the first lamina-spinous process bracket 24, it includes a first lamina-spinous process screw 44 (also referred to herein as the first screw), a first left plate 92, a first right plate 86 (or lamina plates), and a first nut 80. The first lamina-spinous process screw 44 comprises, at least in part, both the first cord securing portion 38 and the first anchoring portion 32, the parts being a first tulip head 56 and a first threaded shaft 50, respectively. The first tulip head 56 includes a first threaded hole 62 formed as a blind axial hole and a first cord slot 68 formed as a through slot across the width of the head 56 and approximately the depth the first threaded hole 62, much like a nock in appearance. The first threaded shaft 50 extends axially from the first tulip head 56 opposite the first threaded hole 62. The cord securing portion 38 in this example embodiment comprises the first threaded hole 62, the first cord slot 68, and a first set screw 74 threadably engageable within the first threaded hole 62. The width and depth of the first cord slot 68 are dimensionally sufficient to hold therewithin a first cord portion 134 of the flexible tensioning cord 22, with the flexible tensioning cord 22 positioned within and extending across the first cord slot 68. To secure the flexible tensioning cord 22 within the first cord slot 68, the first set screw 74 is tightened within the first threaded hole 62 to compress the first cord portion 134 there beneath against the bottom of the first cord slot 68, such that the flexible tensioning cord 22 cannot substantially slide within the first cord slot 68 in this locked configuration. The first left plate 92 includes a first left plate through hole 104 formed through the major face of the first left plate 92 and one or more teeth 110 (hidden in this view) protrude from an inner face and configured to engage a first side of the first posterior boney landmark P1 to prevent rotation of the first left plate 92 relative to the posterior boney landmark P1. The first right plate 86 includes a first right plate through hole 98 formed through the major face of the first right plate 86 and one or more teeth 110 protrude from an inner face and configured to engage a second side of the posterior boney landmark P1 to prevent rotation of the first right plate 86 relative to the posterior boney landmark P1. The inner faces or bone-oriented faces of the first left plate 92 and the first right plate 86 are contoured to more closely fit the contours of the posterior boney landmark P1. The first threaded shaft 50 of the first lamina-spinous process screw 44 is inserted through the first left plate 92 through hole 104 and further through the first right plate 86 through hole 98, which are held on the first threaded shaft 50 by the first nut 80.

Now referencing the illustrated assembly comprising the second lamina-spinous process bracket 26, it includes a second lamina-spinous process screw 46 (also referred to herein as the second screw), a second left plate 94, a second right plate 88, and a second nut 82. The second lamina-spinous process screw 46 comprises, at least in part, both the second cord securing portion 40 and the second anchoring portion 34, the parts being a second tulip head 58 and a second threaded shaft 52, respectively. The second tulip head 58 includes a second threaded hole 64 formed as a blind axial hole and a second cord slot 70 formed as a through slot across the width of the second head 58 and approximately the depth the second threaded hole 64, much like a nock in appearance. The second threaded shaft 52 extends axially from the second tulip head 58 opposite the second threaded hole 64. The second cord securing portion 40 in this example embodiment comprises the second threaded hole 64, the second cord slot 70, and a second set screw 76 threadably engageable within the second threaded hole 64. The width and depth of the second cord slot 70 are dimensionally sufficient to hold therewithin a second cord portion 136 of the flexible tensioning cord 22, with the flexible tensioning cord 22 positioned within and extending across the second cord slot 70. To secure the flexible tensioning cord 22 within the second cord slot 70, the second set screw 76 is tightened within the second threaded hole 64 to compress the second cord portion 136 there beneath against the bottom of the second cord slot 70, such that the flexible tensioning cord 22 cannot substantially slide within the second cord slot 70 in this locked configuration. The second left plate 94 includes a second left plate through hole 106 formed through the major face of the second left plate 94 and one or more teeth 110 (hidden in this view) protrude from an inner face and configured to engage a first side of the second posterior boney landmark P2 to prevent rotation of the second left plate 94 relative to the second posterior boney landmark P2. The second right plate 88 includes a second right plate through hole 100 formed through the major face of the second right plate 88 and one or more teeth 110 protrude from an inner face and configured to engage a second side of the posterior boney landmark P2 to prevent rotation of the second right plate 88 relative to the posterior boney landmark P2. The inner faces or bone-oriented faces of the second left plate 94 and the second right plate 88 are contoured to more closely fit the contours of the posterior boney landmark P2. The second threaded shaft 52 of the second lamina-spinous process screw 46 is inserted through the second left plate 94 through hole 106 and further through the second right plate 88 through hole 100, which are held on the second threaded shaft 52 by the second nut 82.

Finally referencing the illustrated assembly comprising the third lamina-spinous process bracket 28, it includes a third lamina-spinous process screw 48, a third left plate 96, a third right plate 90, and a third nut 82. The third lamina-spinous process screw 48 comprises, at least in part, both the third cord securing portion 42 and the third anchoring portion 36, the parts being a third tulip head 60 and a third threaded shaft 54, respectively. The third tulip head 60 includes a third threaded hole 66 formed as a blind axial hole and a third cord slot 72 formed as a through slot across the width of the third head 60 and approximately the depth the third threaded hole 66, much like a nock in appearance. The third threaded shaft 54 extends axially from the third tulip head 60 opposite the third threaded hole 66. The third cord securing portion 42 in this example embodiment comprises the third threaded hole 66, the third cord slot 72, and a third set screw 78 threadably engageable within the third threaded hole 66. The width and depth of the third cord slot 72 are dimensionally sufficient to hold therewithin a third cord portion 140 of the flexible tensioning cord 22, with the flexible tensioning cord 22 positioned within and extending across the third cord slot 72. To secure the flexible tensioning cord 22 within the third cord slot 72, the third set screw 78 is tightened within the third threaded hole 66 to compress the third cord portion 140 there beneath against the bottom of the third cord slot 72, such that the flexible tensioning cord 22 cannot substantially slide within the third cord slot 72 in this locked configuration. The third left plate 96 includes a third left plate 96 through hole 108 formed through the major face of the third left plate 96 and one or more teeth 110 (hidden in this view) protrude from an inner face and configured to engage a first side of the third posterior boney landmark P3 to prevent rotation of the third left plate 96 relative to the third posterior boney landmark P3. The third right plate 90 includes a third right plate through hole 102 formed through the major face of the third right plate 90 and one or more teeth 110 protrude from an inner face and configured to engage a second side of the third posterior boney landmark P3 to prevent rotation of the third right plate 90 relative to the third posterior boney landmark P3. The inner faces or bone-oriented faces of the third left plate 96 and the third right plate 90 are contoured to more closely fit the contours of the third posterior boney landmark P3. The third threaded shaft 54 of the third lamina-spinous process screw 48 is inserted through the third left plate 96 through hole 108 and further through the third right plate 90 through hole 102, which are held on the third threaded shaft 54 by the second nut 84.

Example embodiments of the tools used for implanting the present fixation system 20, and one or more of which can optionally be a part of the system 20, are illustrated in FIG. 2. A driver tool 33 is used to tighten or loosen the set screws 74, 76, 78, and includes a handle 112, a shaft 114 extending axially from the handle 112, and a bit 116 extending axially from the shaft 114, either permanently attached or exchangeable. Not shown, but well known in the art, are a wrench for holding nuts 80, 82, 84 and a lamina-spinous process screw driver or bit, which may be optionally provided and/or used in the surgical procedure of the present fixation system 20. Further provided, is a tensioning tool 30 including a first handle 118 manually operable relative to a second handle 120 and about pivot 122, where movement of the first handle 118 relative to the second handle 120 causes a bracket grasping jaw half 124 and a cord grasping jaw half 126 to move away from one another, acting as a separator much like pliers or specula in basic operation. The purpose of the tensioning tool 30 is to bear against a portion of one of the lamina-spinous process brackets 24, 26, 28 while pulling the flexible tensioning cord 22 away from that particular lamina-spinous process bracket to provide a therapeutic tension in the flexible tensioning cord. In the illustrated example embodiment, the bracket grasping jaw half 124 is configured to hold the tulip head 56, 58, 60 of one of the lamina-spinous process brackets 24, 26, 28, which would be held within a cradle 128 shaped like an open-ended, hollow hemicylinder sized to cradle therewithin a cylindrical part of the tulip heads 56, 58, 60. The cradle 128 includes an axially aligned slot through a sidewall to provide a cord clearance 132 for the flexible tensioning cord 22 to fit therethrough during a tensioning procedure, creating the appearance of two parallel tines that are positionable on either side of the cord slots 68, 70, 72 of the tulip heads 56, 58, 60. Further, a coaxial hole through the circular end wall of the cradle 128 forms a bit clearance 130, which is sized to permit insertion therethrough or thereabove the bit 116 and/or the shaft 114 of the driver tool during the tensioning procedure. The cord grasping jaw half 126 includes a locking feature 131 configured to grasp the flexible tensioning cord 22 during the tensioning procedure, to prevent substantial slippage of the flexible tensioning cord relative to the locking feature 131. In the illustrated example embodiment, the locking feature 131 includes a longitudinal notch 133 configured to receive therewithin the flexible tensioning cord 22. The longitudinal notch 133 frictionally engages the flexible tensioning cord 22 to prevent slippage. All or a portion of the notch 133 can converge, such that the flexible tensioning cord 22 is pinched and/or wedged therewithin to increase frictional engagement. Other mechanisms to grasp cords, ropes, etc., such as cam locks, clips, and the like, can be used to releasably grasp the flexible tensioning cord 22, so long as the mechanism does not cause substantial damage to the flexible tensioning cord 22 that would results is a substantial change the operational characteristics of the flexible tensioning cord 22.

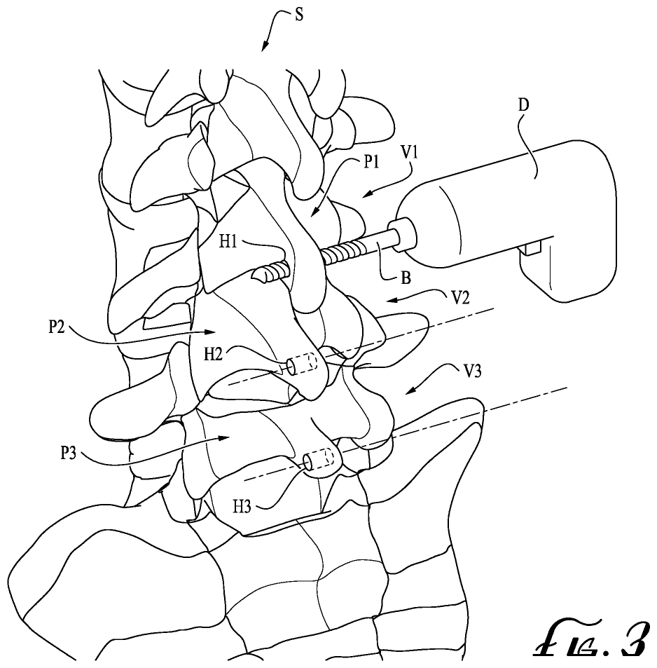

Turning to FIG. 3, which illustrates a portion of a patient's spine S or lamina-spinous process column, which can include the thoracic, lumbar, and sacrum levels. In illustrated example embodiment, a drill D with attached drill bit B is drilling lateral through holes H1, H2, H3 through the posterior boney landmarks P1, P2, P3, specifically the spinous processes SP1, SP2, SP3 of lamina-spinous processes V1, V2, V3 (which are L3, L4, and L5, respectively, of the lumbar spine). Although three adjacent lamina-spinous processes are illustrated as an example, more or fewer adjacent or nonadjacent lamina-spinous processes can be prepared for receiving the present lamina-spinous process brackets.

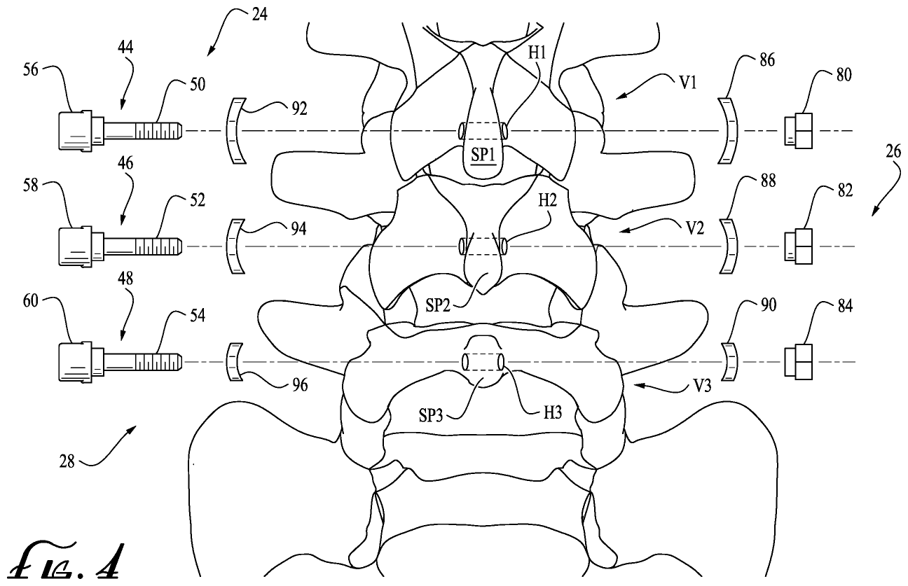

FIG. 4 demonstrates the implanting of the first lamina-spinous process bracket 24 to the first spinous process SP1 of the first lamina-spinous process V1. Here, threaded shaft 50 of lamina-spinous process screw 44 is inserted through first left plate 92 through hole 104, and inserted through hole H1 of spinous process SP1, and though first right plate 86 through hole 98. First nut 80 is thereafter threaded on threaded shaft 50 protruding from first right plate 86, and tightened to an appropriate torque. Although not illustrated, it is understood that a user would hand tighten the nut 80 onto threaded shaft 50, and thereafter using a lamina-spinous process screw driver and a wrench (or other similar tool or tools) the first spinous process SP1 is sandwiched and ultimately clamped between plates 92 and 86, the teeth 110 (not visible in this view) being forced further into engagement to the sides of the first spinous process SP1, while the first nut 80 is tightened to a torque sufficient to prevent unintentional loosening of the nut 80 or substantial movement of the first lamina-spinous process bracket 24 relative to the first spinous process SP1 (or other portion of the posterior boney landmarks P1). With this arrangement, for this and other lamina-spinous process brackets, the first head 56 extends laterally toward the patient's left side. However, the first head 56 can be positioned oppositely to extend to the patient's right side. Further, although hole H1 is illustrated as being drilled laterally through the spinous process, the hole can be drilled though various intact parts of the posterior boney landmark, such as the lamina or the junction of the lamina and spinous process, which could change the orientation of the hole H1 and the first lamina-spinous process screw 44 inserted therethrough.

Still viewing FIG. 4, and like the implanting of the first lamina-spinous process bracket 24 described above, the second lamina-spinous process bracket 26 is being implanted to the second spinous process SP2 of the second lamina-spinous process V2. Here, threaded shaft 52 of lamina-spinous process screw 46 is inserted through second left plate 94 through hole 106, and inserted through hole H2 of spinous process SP2, and though second right plate 88 through hole 100. Second nut 82 is thereafter threaded on threaded shaft 52 protruding from second right plate 88, and tightened to an appropriate torque. The nut 82 is hand-tightened onto threaded shaft 52, and thereafter using a lamina-spinous process screw driver and a wrench the second spinous process SP2 is sandwiched and ultimately clamped between plates 94 and 88, the teeth 110 (not visible in this view) being forced further into engagement to the sides of the second spinous process SP2, while the second nut 82 is tightened to a torque sufficient to prevent unintentional loosening of the nut 82 or substantial movement of the second lamina-spinous process bracket 26 relative to the second spinous process SP2 (or other portion of the posterior boney landmarks P2).

And like the implanting of the first lamina-spinous process bracket 24 described above, the third lamina-spinous process bracket 28 is being implanted to the third spinous process SP3 of the third lamina-spinous process V3. Here, threaded shaft 54 of lamina-spinous process screw 48 is inserted through third left plate 96 through hole 108, and inserted through hole H3 of spinous process SP3, and though third right plate 90 through hole 102. Third nut 84 is thereafter threaded on threaded shaft 54 protruding from third right plate 90, and tightened to an appropriate torque. The nut 84 is hand-tightened onto threaded shaft 54, and thereafter using a lamina-spinous process screw driver and a wrench the third spinous process SP3 is sandwiched and ultimately clamped between plates 96 and 90, the teeth 110 (not visible in this view) being forced further into engagement to the sides of the third spinous process SP3, while the third nut 84 is tightened to a torque sufficient to prevent unintentional loosening of the nut 84 or substantial movement of the third lamina-spinous process bracket 28 relative to the third spinous process SP3 (or other portion of the posterior boney landmarks P3).

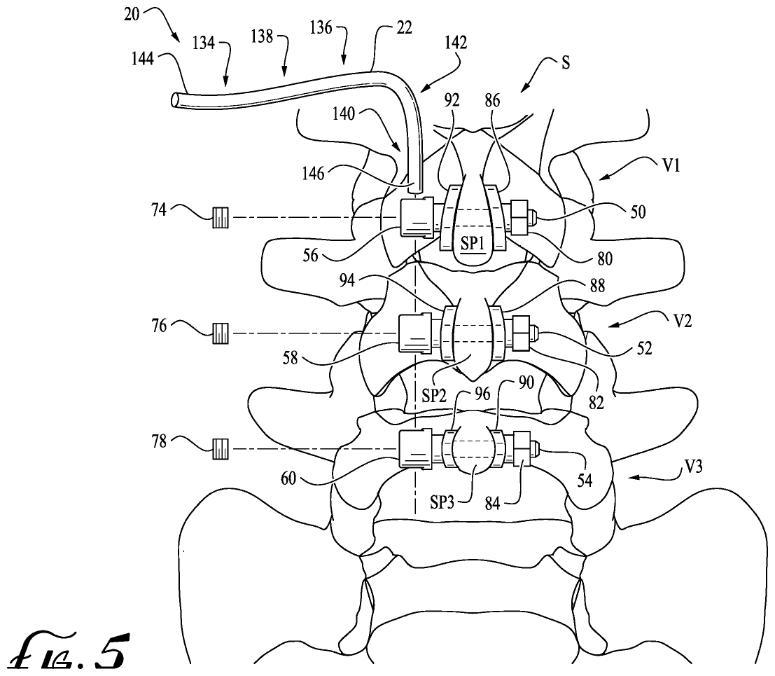

Once the fixation system 20 is assembled partially (where each of the first lamina-spinous process bracket 24, the second lamina-spinous process bracket 26, and the third lamina-spinous process bracket 28 are securely coupled to their respective lamina-spinous process), as shown in FIG. 4, the flexible tensioning cord 22 can be attached thereto. Looking at FIG. 5, the flexible tensioning cord 22 can be placed within one or more of the cord slots 68, 70, 72. In one example assembly method, the first cord portion 134 is placed within the first cord slot 68 with the first free end 144 of the flexible tensioning cord 22 situated beyond and superior to the first tulip head 56, the second cord portion 136 is placed within the second cord slot 70 with connecting portion 138 extending between the first tulip head 56 and the second tulip head 58, and the third cord portion 140 is placed within the third cord slot 72 with connecting portion 142 extending between the second tulip head 58 and the third tulip head 60. The set screws 74, 76, 78 can be partially threaded into their respective threaded holes 62, 64, 66 at any appropriate time as the flexible tensioning cord 22 is being placed in each cord slots 68, 70, 72.

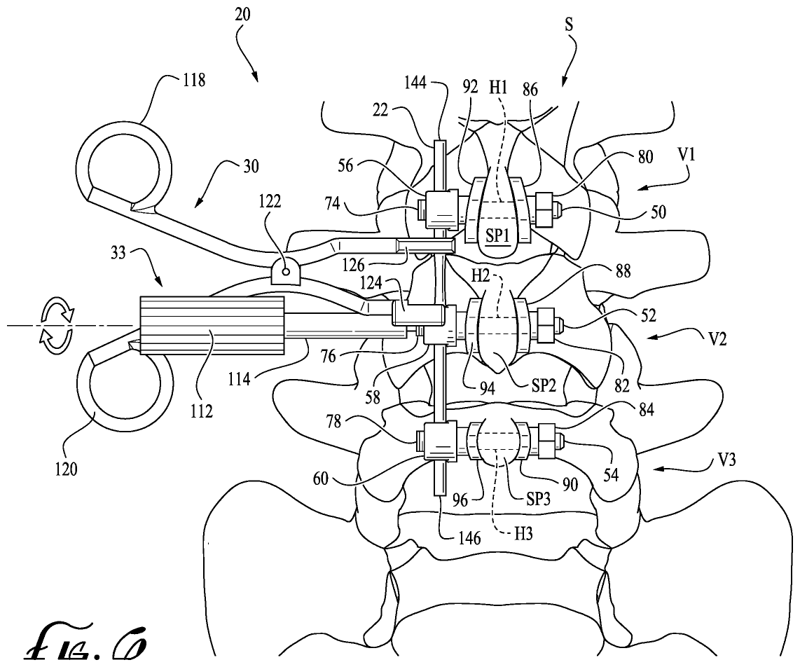

Generally, during the tensioning procedure, it is desirable to leave one or more of the set screws 74, 76, 78 sufficiently loose to permit the flexible tensioning cord 22 to slide within one or more cord slots 68, 70, 72. In a first exemplary step, as seen in FIG. 6, third set screw 78 is tightened within the third threaded hole 66, clamping the third cord portion 140 between the third set screw 78 and the bottom of the third threaded hole 66 to prevent substantial sliding of the flexible tensioning cord 22 relative to the third cord slot 72. The first and third set screw 76, 78 remain loose, such that the flexible tensioning cord 22 can slide relative to at least the second cord slot 70 and optionally the first cord slot 68. With the third set screw 78 tight, the bracket grasping jaw half 124 of the tensioning tool 30 is releasably applied or coupled to the second tulip head 58 and the cord grasping jaw half 124 is releasably coupled to connecting portion 138 of the flexible tensioning cord 22. As the operator manually moves handle 118 toward handle 120, the cord grasping jaw half 124 is forced away from the bracket grasping jaw half 124 due to the bracket grasping jaw half 124 bearing down on the relatively immovable second lamina-spinous process bracket 26. As the cord grasping jaw half 124 moves upward pulling flexible tensioning cord 22 along, the tension between the cord grasping jaw half 124 and the first lamina-spinous process bracket 24 increases to a therapeutic tension force magnitude. While the tensioning tool 30 is held in place, imparting the therapeutic tension force on this portion of the flexible tensioning cord 22, the operator inserts the one or both of the bit 116 and the shaft 114 of the driver tool 33 through the bit clearance 130 of the bracket grasping jaw half 124, engaging the second set screw 76, and tightening the second set screw 76 by twisting handle 112. Once the second set screw 76 is tightened to firmly hold the flexible tensioning cord 22, the driver tool 33 and the tensioning tool 30 are both removed, leaving connecting portion 142 of the flexible tensioning cord 22 within tolerance of the desired therapeutic tension between the second set screw 76 and the third set screw 78.

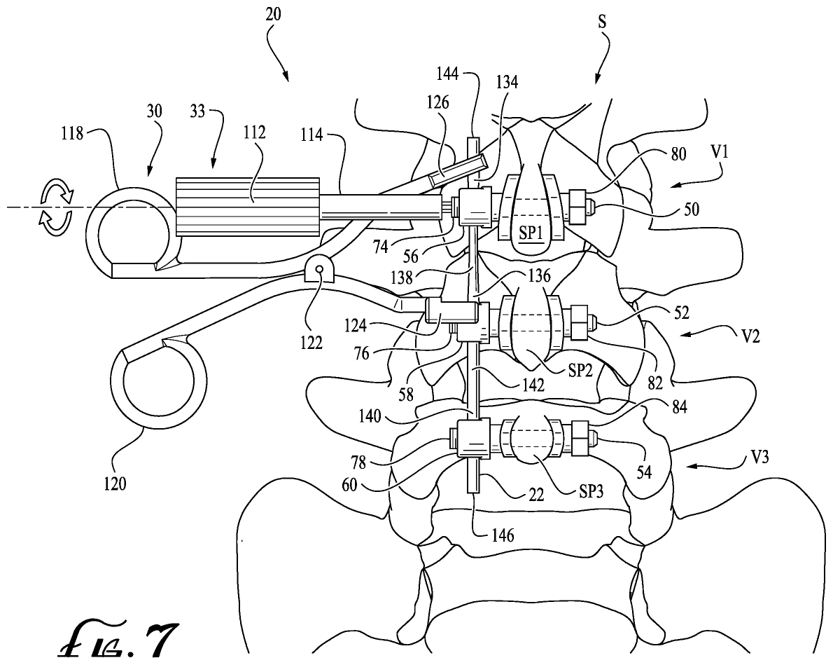

The tensioning procedure is continued in FIG. 7, and like the procedure of FIG. 6, set screw 74 is tightened while the tensioning tool 30 maintains the connecting portion 138 of the flexible tensioning cord 22 at the desired therapeutic tension, which may be substantially the same tension as above or a differing tension. In the tensioning procedure, the bracket grasping jaw half 124 bears against the tulip head 58 of the second lamina-spinous process bracket 26, while the cord grasping jaw half 124 is releasably coupled to the flexible tensioning cord 22 between the free end 144 and the first set screw 74 of the first tulip head 56. As in the procedure of FIG. 6, the operator squeezes handles 118 and 120 together to expand the jaws of the tensioning tool 30, thus moving the cord grasping jaw half 124 away from the bracket grasping jaw half 124 to tension the connecting portion 138 of the flexible tensioning cord 22. While held in tension, the operator tightens the first set screw 74 with the driver tool 33, leaving connecting portion 138 of the flexible tensioning cord 22 within tolerance of the desired therapeutic tension between the second set screw 76 and the first set screw 74. In one or more example embodiments, the therapeutic tension is a tension that induces lordosis in the spine of a particular patient, which can vary from patient to patient. In one or more example embodiments, minimal or zero tension can be set in one or more connecting portions.

Although, the bracket grasping jaw half 124 is illustrated as bearing against the second tulip head 58 in both FIGS. 6 and 7, it should be understood that the design of the present tensioning tool 30 permits the bracket grasping jaw half 124 to be placed on any one of the tulip heads 56, 58, 60 to tighten the region of the flexible tensioning cord 22 between a tightened set screw and the cord grasping jaw half 124 (with no intervening tightened set screw). This flexibility permits the operator to rearrange purchase from one tulip head to another (or other part of the lamina-spinous process brackets) depending on access, leverage, ergonomics, and the width of the jaw opening. Further, the tensioning tool 33 can be modified in use or construction to bear against or otherwise be releasably coupled to other parts of the lamina-spinous process brackets 24, 26, 28 or even a portion of the lamina-spinous process. Further, although the exemplary tensioning procedure is illustrated in a particular order of tensioning, in use, the order may vary according to the requirements of the procedure.

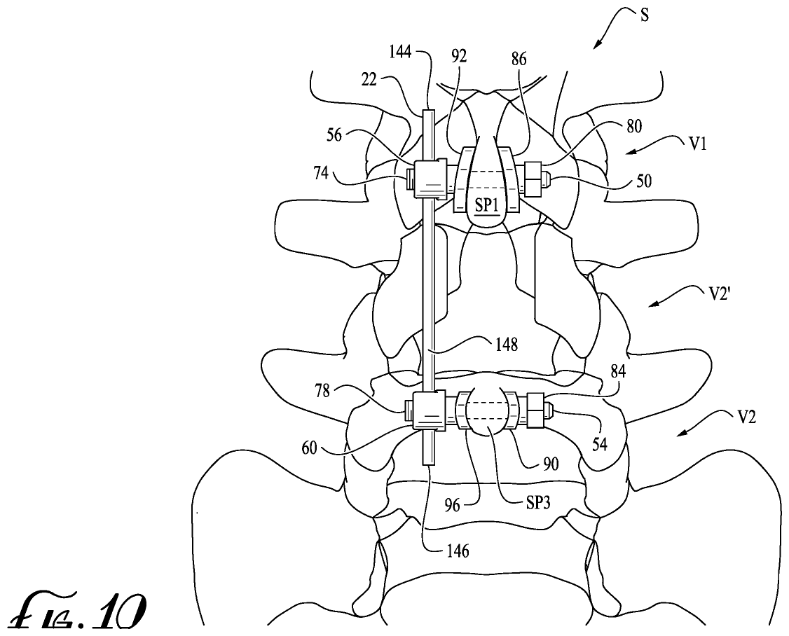

FIGS. 8 and 9 illustrate the first lamina-spinous process bracket 24, the second lamina-spinous process bracket 26, the third lamina-spinous process bracket 28, and the flexible tensioning cord 22 implanted to the spine S of a patient, with a first tension set in connecting portion 138 and a second tension set in connecting portion 142. Of course, as the patient moves, the tensions can vary, yet will provide bias to return the curvature of the spine S to the degree of lordosis set in the procedure. If slight permanent stretching or loosening is expected in the flexible tensioning cord 22 (which is made of flexible polyethylene-terephthalate (PET), for example, sold under the name SULENE, a multifilament, braided polyester cord), a slightly higher initial tension may be set to compensate for later changes. Although three lamina-spinous process brackets 24, 26, 28 are illustrated, more or fewer can be used. Moreover, although the present implanted assembly illustrates the flexible tensioning cord 22 being tensioned between two lamina-spinous process brackets, the flexible tensioning cord 22 can alternatively be coupled between a single lamina-spinous process bracket and directly to a posterior boney landmark or other anchoring device attached to the posterior boney landmark.

Often, a full or partial laminectomy is performed prior to implanting the present assembly of one or more lamina-spinous process brackets to ease pressure on the spinal cord, thecal sac, or spinal nerves. In that case, the lamina-spinous process may have insufficient posterior boney structure to support a lamina-spinous process bracket. FIG. 10 illustrates a patient's spine S in which a laminectomy has been performed on lamina-spinous process V2′. In order to provide support, and rather than performing a spinal fusion procedure, the first lamina-spinous process bracket 24 is mounted to the first spinous process SP1 and the second lamina-spinous process bracket 26 is mounted to the second spinous process SP2, where the lamina-spinous process V1 and lamina-spinous process V2 are nonconsecutive levels with lamina-spinous process V2′ positioned therebetween. As described above, the flexible tensioning cord 22 is anchored between the first lamina-spinous process bracket 24 and the third lamina-spinous process bracket 28, with connecting portion 148 extending over lamina-spinous process V2′.

A kit of the present system 20 can minimally include a first lamina-spinous process bracket 24 assembly and a flexible tensioning cord. The kit can further include a second lamina-spinous process bracket 26. The kit can further include a tensioning tool 30 and/or a driver tool 33. The kit can further include a drill bit. The kit can further include a wrench.

Aspects of the present specification may also be described as follows:

A fixation system for restoring a posterior tension band in a spine of a patient is provided, the fixation system having a flexible tensioning cord, a first lamina-spinous process backet, a second lamina-spinous process bracket, and a tensioning tool. The flexible tensioning cord has a first cord portion, a second cord portion, and a connecting portion extending between the first cord portion and the second cord portion; the first lamina-spinous process bracket has a first anchoring portion and a first cord securing portion adapted to permit the flexible tensioning cord to slide therethrough in an open configuration and adapted to securely grasp the flexible tensioning cord at the first cord portion in a locked configuration, the first lamina-spinous process bracket adapted to be anchored to a first posterior boney landmark of the spine by the first anchoring portion; the second lamina-spinous process bracket has a second anchoring portion and a second cord securing portion adapted to permit the flexible tensioning cord to slide therethrough in the open configuration and adapted to securely grasp the flexible tensioning cord at the second cord portion in the locked configuration, the second lamina-spinous process bracket adapted to be anchored to a second posterior boney landmark of the spine by the second anchoring portion; and the tensioning tool having a bracket grasping jaw half opposing a cord grasping jaw half. When in a tensioning configuration, the first lamina-spinous process bracket is in the locked configuration, the second lamina-spinous process bracket is in the open configuration, the bracket grasping jaw half of the tensioning tool is releasably coupled to the first lamina-spinous process bracket and the cord grasping jaw half is releasably coupled to the flexible tensioning cord to hold the connecting portion in tension while the second lamina-spinous process bracket is reconfigured to the locked configuration, holding therebetween the flexible tensioning cord at a therapeutic tension.

In one or more embodiments, the tensioning tool further includes a first handle in connection with the bracket grasping jaw half and a second handle in connection with the cord grasping jaw half, the movement of the first handle relative to the second handle causes separation of the cord grasping jaw half relative to the bracket grasping jaw half.

In one or more embodiments, the bracket grasping jaw half is a cradle configured to bear against the first lamina-spinous process bracket in the tensioning configuration, the cord grasping jaw half is a locking feature within which the connecting portion of the flexible tensioning cord is gripped in the tensioning configuration, each of the cradle and the locking feature are selectively separable from the first lamina-spinous process bracket and the flexible tensioning cord.

In one or more embodiments, a driver is provided and is configured to transition the first lamina-spinous process bracket and the second lamina-spinous process bracket between the open configuration and the locked configuration.

In one or more embodiments, the first lamina-spinous process bracket is substantially similar to the second lamina-spinous process bracket, where the first lamina-spinous process bracket includes a lamina-spinous process screw having a threaded shaft connected to a tulip head and a set screw threadably engaged within the first tulip-head; the first anchoring portion including the threaded shaft, the nut, a right plate having a right through hole, and a left plate having a left through hole; and the first cord securing portion comprising the tulip-head and a set screw threadably engaged within the tulip-head through which the flexible tensioning cord is selectively threaded and clamped therebetween. Where, when in an assembled configuration, the right plate is configured to engage a right side of the posterior boney landmark and the left plate is configured to engage a left side of the posterior boney landmark with the right through hole and the left through hole configured to be aligned with a posterior boney landmark hole, with the threaded shaft of the lamina-spinous process screw inserted through each of the right through hole, the posterior boney landmark hole, and the left through hole, the nut threadably engaged on the threaded shaft of the lamina-spinous process screw and configured to clamp the posterior boney landmark between the right plate and the left plate.

In one or more embodiments, the flexible tensioning cord is a polyethylene-terephthalate cord.

A method to induce lordosis in a spine of a patient includes the steps of anchoring a first bracket to a first posterior boney landmark of a first lamina-spinous process of the spine, the first bracket having a first cord securing portion; anchoring a second bracket to a second posterior boney landmark of a second lamina-spinous process of the spine, the second bracket having a second cord securing portion; securing a flexible tensioning cord to the first securing portion of the first bracket by a first cord portion, the flexible tensioning cord having a second cord portion, and a connecting portion extending between the first cord portion and the second cord portion; bearing a bracket grasping jaw half of a tensioning tool against the first bracket, a cord grasping jaw half of the tensioning tool opposing the bracket grasping jaw half; coupling the cord grasping jaw half with the flexible tensioning cord; applying a tensioning force to the flexible tensioning cord by manipulation of the tensioning tool to move the bracket grasping jaw half away from the cord grasping jaw half; and securing the flexible tensioning cord, during application of the tensioning force, to the second securing portion of the second lamina-spinous process bracket by a second cord portion to maintain a lordosis inducing tension in the flexible tensioning cord.

In one or more embodiments, a third lamina-spinous process is situated between the first lamina-spinous process and the second lamina-spinous process, where a laminectomy has been performed on the third lamina-spinous process.

In one or more embodiments, the first posterior boney landmark of the first lamina-spinous process is a first spinous process and/or a first lamina and the second posterior boney landmark of the second lamina-spinous process is a second spinous process and/or a second lamina.

In one or more embodiments, the first bracket includes a first screw having a first tulip-head and a first threaded shaft threadably engageable to a first nut, the first cord securing portion comprising the first tulip-head and a first set screw threadably engageable therewithin and through which the flexible tensioning cord is selectively threaded; and the second bracket includes a second screw having a second tulip-head and a second threaded shaft threadably engageable to a second nut, the second cord securing portion comprising the second tulip-head and a second set screw threadably engageable therewithin and through which the flexible tensioning cord is selectively threaded; a first right plate includes a first right through hole and a first left plate having a first left through hole; and a second right plate includes a second right through hole and a second left plate having a second left through hole.

In one or more embodiments the anchoring of a first bracket to a first posterior boney landmark further includes drilling a first hole through a first posterior boney landmark; engaging the first right plate to a first right side of the first posterior boney landmark and the first left plate to a first left side of the first posterior boney landmark with the first right through hole, the first left through hole, and the first hole aligned; inserting the first threaded shaft through the first right through hole, the first left through hole, and the first hole; engaging the first nut on the first threaded shaft with sufficient torque to anchor the first bracket to the first posterior boney landmark; drilling a second hole through a second posterior boney landmark; engaging the second right plate to a second right side of the second posterior boney landmark and the second left plate to a second left side of the second posterior boney landmark with the second right through hole, the second left through hole, and the second hole aligned; inserting the second threaded shaft through the second right through hole, the second left through hole, and the second hole; and engaging the second nut on the second threaded shaft with sufficient torque to anchor the second bracket to the second posterior boney landmark.

In one or more embodiments, the bracket grasping jaw half is a cradle and the cord grasping jaw half is a locking feature within which the connecting portion of the flexible tensioning cord is temporarily wedged.

A method for implanting a prosthesis for restoring a posterior tension band in a spine of a patient is provided and includes using a fixation system comprising a first bracket, a second bracket, a flexible tensioning cord, and a tensioning tool, the first bracket having a first anchoring portion and a first cord securing portion, the second bracket having a second anchoring portion and a second cord securing portion, the flexible tensioning cord having a first cord portion, a second cord portion, and a connecting portion extending therebetween, and the tensioning tool having a bracket grasping jaw half opposing a cord grasping jaw half; anchoring the first bracket by the first anchoring portion to a first posterior boney landmark of a first lamina-spinous process of the spine; anchoring the second bracket by the second anchoring portion to a second posterior boney landmark of a second lamina-spinous process of the spine; securing a flexible tensioning cord to the first securing portion of the first bracket by the first cord portion; bearing the bracket grasping jaw half of the tensioning tool against the first bracket; coupling the cord grasping jaw half with the flexible tensioning cord; applying a tensioning force to the flexible tensioning cord by manipulation of the tensioning tool to separate the cord grasping jaw half from the bracket grasping jaw half; and securing the flexible tensioning cord, during application of the tensioning force, to the second securing portion of the second bracket by a second cord portion to maintain a lordosis inducing tension in the flexible tensioning cord between the first bracket and the second bracket.

In one or more embodiments, a third lamina-spinous process is situated between the first lamina-spinous process and the second lamina-spinous process, where a laminectomy has been performed on the third lamina-spinous process.

In one or more embodiments, the first posterior boney landmark of the first lamina-spinous process is a first spinous process and/or a first lamina and the second posterior boney landmark of the second lamina-spinous process is a second spinous process and/or a second lamina.

In one or more embodiments, the first bracket includes a first screw having a first tulip-head and a first threaded shaft threadably engageable to a first nut, the first cord securing portion comprising the first tulip-head and a first set screw threadably engageable therewithin and through which the flexible tensioning cord is selectively threaded; and the second bracket includes a second screw having a second tulip-head and a second threaded shaft threadably engageable to a second nut, the second cord securing portion comprising the second tulip-head and a second set screw threadably engageable therewithin and through which the flexible tensioning cord is selectively threaded; a first right plate includes a first right through hole and a first left plate having a first left through hole; and a second right plate includes a second right through hole and a second left plate having a second left through hole.

In one or more embodiments, anchoring the first bracket to a first posterior boney landmark further includes drilling a first hole through a first posterior boney landmark; engaging the first right plate to a first right side of the first posterior boney landmark and the first left plate to a first left side of the first posterior boney landmark with the first right through hole, the first left through hole, and the first hole aligned; inserting the first threaded shaft through the first right through hole, the first left through hole, and the first hole; engaging the first nut on the first threaded shaft with sufficient torque to anchor the first bracket to the first posterior boney landmark; drilling a second hole through a second posterior boney landmark; engaging the second right plate to a second right side of the second posterior boney landmark and the second left plate to a second left side of the second posterior boney landmark with the second right through hole, the second left through hole, and the second hole aligned; inserting the second threaded shaft through the second right through hole, the second left through hole, and the second hole; and engaging the second nut on the second threaded shaft with sufficient torque to anchor the second bracket to the second posterior boney landmark.

In one or more embodiments, the bracket grasping jaw half is a cradle and the cord grasping jaw half is a locking feature within which the connecting portion of the flexible tensioning cord is temporarily wedged.

In closing, it is to be understood that, although aspects of the present specification are highlighted by referring to specific embodiments, one skilled in the art will readily appreciate that these disclosed embodiments are only illustrative of the principles of the subject matter disclosed herein. The specific embodiments are not intended to be exhaustive or to limit the invention to the precise forms disclosed. Therefore, it should be understood that the disclosed subject matter is in no way limited to a particular compound, composition, article, apparatus, methodology, and/or protocol, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the scope of the invention is not to be limited by this detailed description. Furthermore, it is intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

Unless otherwise indicated, all numbers expressing a characteristic, item, quantity, parameter, property, term, and so forth used in the present specification and claims are to be understood as being modified in all instances by the term “about” and/or “approximately.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including,” “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Thus, the meaning of the open-ended transitional phrase “comprising” is being defined as encompassing all the specifically recited elements, limitations, steps and/or features as well as any optional, additional unspecified ones.

Lastly, the terminology used herein is for the purpose of describing particular embodiments only and is not intended to limit the scope of the present invention, which is defined solely by the claims. Accordingly, the present invention is not limited to that precisely as shown and described.

What is claimed is:

1) A fixation system for restoring a posterior tension band in a spine of a patient, the fixation system comprising:
a flexible tensioning cord having a first cord portion, a second cord portion, and a connecting portion extending between the first cord portion and the second cord portion;
a first lamina-spinous process bracket having a first anchoring portion and a first cord securing portion adapted to permit the flexible tensioning cord to slide therethrough in an open configuration and adapted to securely grasp the flexible tensioning cord at the first cord portion in a locked configuration, the first lamina-spinous process bracket adapted to be anchored to a first posterior boney landmark of the spine by the first anchoring portion;
a second lamina-spinous process bracket having a second anchoring portion and a second cord securing portion adapted to permit the flexible tensioning cord to slide therethrough in the open configuration and adapted to securely grasp the flexible tensioning cord at the second cord portion in the locked configuration, the second lamina-spinous process bracket adapted to be anchored to a second posterior boney landmark of the spine by the second anchoring portion; and
a tensioning tool having a bracket grasping jaw half opposing a cord grasping jaw half;
wherein, in a tensioning configuration, the first lamina-spinous process bracket is in the locked configuration, the second lamina-spinous process bracket is in the open configuration, the bracket grasping jaw half of the tensioning tool is releasably coupled to the first lamina-spinous process bracket and the cord grasping jaw half is releasably coupled to the flexible tensioning cord to hold the connecting portion in tension while the second lamina-spinous process bracket is reconfigured to the locked configuration, holding therebetween the flexible tensioning cord at a therapeutic tension.

2) The fixation system of claim 1 wherein the tensioning tool further comprises a first handle in connection with the bracket grasping jaw half and a second handle in connection with the cord grasping jaw half, the movement of the first handle relative to the second handle causes separation of the cord grasping jaw half relative to the bracket grasping jaw half.

3) The fixation system of claim 1 wherein the bracket grasping jaw half is a cradle configured to bear against the first lamina-spinous process bracket in the tensioning configuration, the cord grasping jaw half is a locking feature within which the connecting portion of the flexible tensioning cord is gripped in the tensioning configuration, each of the cradle and the locking feature are selectively separable from the first lamina-spinous process bracket and the flexible tensioning cord.

4) The fixation system of claim 1 further comprising a driver configured to transition the first lamina-spinous process bracket and the second lamina-spinous process bracket between the open configuration and the locked configuration.

5) The fixation system of claim 1 wherein the first lamina-spinous process bracket is substantially similar to the second lamina-spinous process bracket, the first lamina-spinous process bracket comprising:
a lamina-spinous process screw having a threaded shaft connected to a tulip head and a set screw threadably engaged within the first tulip-head;
the first anchoring portion comprising the threaded shaft, the nut, a right plate having a right through hole, and a left plate having a left through hole; and
the first cord securing portion comprising the tulip-head and a set screw threadably engaged within the tulip-head through which the flexible tensioning cord is selectively threaded and clamped therebetween;
wherein, in an assembled configuration, the right plate is configured to engage a right side of the posterior boney landmark and the left plate is configured to engage a left side of the posterior boney landmark with the right through hole and the left through hole configured to be aligned with a posterior boney landmark hole, with the threaded shaft of the lamina-spinous process screw inserted through each of the right through hole, the posterior boney landmark hole, and the left through hole, the nut threadably engaged on the threaded shaft of the lamina-spinous process screw and configured to clamp the posterior boney landmark between the right plate and the left plate.

6) The fixation system of claim 1 wherein the flexible tensioning cord is a polyethylene-terephthalate cord.

7) A method to induce lordosis in a spine of a patient, the method comprising:
anchoring a first bracket to a first posterior boney landmark of a first lamina-spinous process of the spine, the first bracket having a first cord securing portion;
anchoring a second bracket to a second posterior boney landmark of a second lamina-spinous process of the spine, the second bracket having a second cord securing portion;
securing a flexible tensioning cord to the first securing portion of the first bracket by a first cord portion, the flexible tensioning cord having a second cord portion, and a connecting portion extending between the first cord portion and the second cord portion;
bearing a bracket grasping jaw half of a tensioning tool against the first bracket, a cord grasping jaw half of the tensioning tool opposing the bracket grasping jaw half;
coupling the cord grasping jaw half with the flexible tensioning cord;
applying a tensioning force to the flexible tensioning cord by manipulation of the tensioning tool to move the bracket grasping jaw half away from the cord grasping jaw half; and
securing the flexible tensioning cord, during application of the tensioning force, to the second securing portion of the second bracket by a second cord portion to maintain a lordosis inducing tension in the flexible tensioning cord.

8) The method of claim 7 wherein a third lamina-spinous process is situated between the first lamina-spinous process and the second lamina-spinous process.

9) The method of claim 7 wherein a third lamina-spinous process is situated between the first lamina-spinous process and the second lamina-spinous process, the method further comprising:
performing a laminectomy on the third lamina-spinous process.

10) The method of claim 7 wherein the first posterior boney landmark of the first lamina-spinous process is a first spinous process and/or a first lamina and the second posterior boney landmark of the second lamina-spinous process is a second spinous process and/or a second lamina.

11) The method of claim 7 wherein:
the first bracket comprises a first screw having a first tulip-head and a first threaded shaft threadably engageable to a first nut, the first cord securing portion comprising the first tulip-head and a first set screw threadably engageable therewithin and through which the flexible tensioning cord is selectively threaded; and
the second bracket comprises a second screw having a second tulip-head and a second threaded shaft threadably engageable to a second nut, the second cord securing portion comprising the second tulip-head and a second set screw threadably engageable therewithin and through which the flexible tensioning cord is selectively threaded;
a first right plate having a first right through hole and a first left plate having a first left through hole; and
a second right plate having a second right through hole and a second left plate having a second left through hole.

12) The method of claim 11 wherein anchoring the first bracket to the first posterior boney landmark further comprises:
drilling a first hole through the first posterior boney landmark;
engaging the first right plate to a first right side of the first posterior boney landmark and the first left plate to a first left side of the first posterior boney landmark with the first right through hole, the first left through hole, and the first hole aligned;
inserting the first threaded shaft through the first right through hole, the first left through hole, and the first hole;
engaging the first nut on the first threaded shaft with sufficient torque to anchor the first bracket to the first posterior boney landmark;
drilling a second hole through a second posterior boney landmark;
engaging the second right plate to a second right side of the second posterior boney landmark and the second left plate to a second left side of the second posterior boney landmark with the second right through hole, the second left through hole, and the second hole aligned;
inserting the second threaded shaft through the second right through hole, the second left through hole, and the second hole; and
engaging the second nut on the second threaded shaft with sufficient torque to anchor the second bracket to the second posterior boney landmark.

13) The method of claim 7 wherein the bracket grasping jaw half is a cradle and the cord grasping jaw half is a locking feature within which the connecting portion of the flexible tensioning cord is temporarily wedged.

14) A method for implanting a prosthesis for restoring a posterior tension band in a spine of a patient, the method comprising:
using a fixation system comprising a first bracket, a second bracket, a flexible tensioning cord, and a tensioning tool, the first bracket having a first anchoring portion and a first cord securing portion, the second bracket having a second anchoring portion and a second cord securing portion, the flexible tensioning cord having a first cord portion, a second cord portion, and a connecting portion extending therebetween, and the tensioning tool having a bracket grasping jaw half opposing a cord grasping jaw half;
anchoring the first bracket by the first anchoring portion to a first posterior boney landmark of a first lamina-spinous process of the spine;
anchoring the second bracket by the second anchoring portion to a second posterior boney landmark of a second lamina-spinous process of the spine;
securing a flexible tensioning cord to the first securing portion of the first bracket by the first cord portion;
bearing the bracket grasping jaw half of the tensioning tool against the first bracket;
coupling the cord grasping jaw half with the flexible tensioning cord;
applying a tensioning force to the flexible tensioning cord by manipulation of the tensioning tool to separate the cord grasping jaw half from the bracket grasping jaw half; and
securing the flexible tensioning cord, during application of the tensioning force, to the second securing portion of the second bracket by a second cord portion to maintain a lordosis inducing tension in the flexible tensioning cord between the first bracket and the second bracket.

15) The method of claim 14 wherein a third lamina-spinous process is situated between the first lamina-spinous process and the second lamina-spinous process.

16) The method of claim 14 wherein a third lamina-spinous process is situated between the first lamina-spinous process and the second lamina-spinous process, the method further comprising:
performing a laminectomy on the third lamina-spinous process.

17) The method of claim 14 wherein the first posterior boney landmark of the first lamina-spinous process is a first spinous process and/or a first lamina and the second posterior boney landmark of the second lamina-spinous process is a second spinous process and/or a second lamina.

18) The method of claim 14 wherein:
the first bracket comprises a first screw having a first tulip-head and a first threaded shaft threadably engageable to a first nut, the first cord securing portion comprising the first tulip-head and a first set screw threadably engageable therewithin and through which the flexible tensioning cord is selectively threaded; and
the second bracket comprises a second screw having a second tulip-head and a second threaded shaft threadably engageable to a second nut, the second cord securing portion comprising the second tulip-head and a second set screw threadably engageable therewithin and through which the flexible tensioning cord is selectively threaded;
a first right plate having a first right through hole and a first left plate having a first left through hole; and
a second right plate having a second right through hole and a second left plate having a second left through hole.

19) The method of claim 18 wherein the anchoring the first bracket to the first posterior boney landmark further comprises:
drilling a first hole through the first posterior boney landmark;
engaging the first right plate to a first right side of the first posterior boney landmark and the first left plate to a first left side of the first posterior boney landmark with the first right through hole, the first left through hole, and the first hole aligned;
inserting the first threaded shaft through the first right through hole, the first left through hole, and the first hole;
engaging the first nut on the first threaded shaft with sufficient torque to anchor the first bracket to the first posterior boney landmark;
drilling a second hole through a second posterior boney landmark;
engaging the second right plate to a second right side of the second posterior boney landmark and the second left plate to a second left side of the second posterior boney landmark with the second right through hole, the second left through hole, and the second hole aligned;
inserting the second threaded shaft through the second right through hole, the second left through hole, and the second hole; and
engaging the second nut on the second threaded shaft with sufficient torque to anchor the second bracket to the second posterior boney landmark.

20) The method of claim 14 wherein the bracket grasping jaw half is a cradle and the cord grasping jaw half is a locking feature within which the connecting portion of the flexible tensioning cord is temporarily wedged.
